(19) United States
(12) Patent Application Publication
(10) Pub. No.: US 2015/0238698 A1
(43) Pub. Date: Aug. 27, 2015

(54) ASPIRATION SYRINGE ACCESSORY

(71) Applicant: Allergan, Inc., Irvine, CA (US)

(72) Inventors: Robert W. Perry, Santa Barbara, CA (US); Edwin J. Kayda, Santa Barbara, CA (US); Justin J. Schwab, Santa Barbara, CA (US)

(73) Assignee: Allergan, Inc., Irvine, CA (US)

(21) Appl. No.: 14/192,609

(22) Filed: Feb. 27, 2014

(51) Int. Cl. A61M 5/31; A61M 5/315

(52) U.S. Cl. CPC A61M 5/3137; A61M 5/31511; A61M 2005/3139; A61M 2202/0007; A61M 2210/04

(57) ABSTRACT

A device is provided for facilitating an aspiration procedure during a dermal filler injection treatment. The device can be connected to a dermal filler syringe plunger, and includes a thumb stop element for engaging a physician's thumb.

The present invention generally relates to syringes for injection of dermal fillers, and more specifically to a modified dermal filler syringe or an accessory for a dermal filler syringe.

Injectable dermal fillers have been proven to be safe and effective for treating soft tissue conditions, for example, for improving the appearance of the aging face. When skillfully introduced into or beneath the skin, these materials can effectively plump facial skin and redefine the lips, correct wrinkles, folds, creases and even fine lines, add volume back to sunken cheeks, and reduce the appearance of surgical scars, acne divots and other imperfections. A polymer commonly used in dermal filler compositions is hyaluronan, or hyaluronic acid. Hyaluronan is found naturally and abundantly in the human body. There are numerous, commercially available, dermal fillers comprising chemically crosslinked hyaluronan, which provide excellent aesthetic treatment results for several months up to a year after injection.

Naturally, the needle used for dermal filler injection is preferred to be quite fine, at least for purposes of minimizing pain, but also for facilitating greater precision in treatment of fine lines in the dermis. Although most commercial dermal fillers are considered safe and are physiologically well tolerated, a rare complication that may occur during treatment, if proper precaution is not taken, is the introduction of the filler into a blood vessel. It is therefor recommended that physicians, when injecting a dermal filler, first “aspirate” the syringe, to ensure the needle tip is not located within a blood vessel, prior to injecting the product.

Aspiration is typically performed by the physician/injector by first inserting needle tip into the skin at the desired injection site, and, while using a free hand to hold the syringe and keep the needle position still, using the other hand pull back on the syringe plunger. If the physician observes that blood is pulled into the syringe, this indicates that the needle tip may be located in a vessel, and should be removed and repositioned. The physician can then move the needle tip to a different location, and repeat the aspiration procedure. When the physician does not see blood aspirated into the syringe upon withdrawal of the syringe plunger, he can then proceed to press the plunger in the distal direction and safely inject the dermal filler.

It can be appreciated that the aspiration procedure can be cumbersome in that it requires changing of the physician's grip. Although this may not seem to be problematic in itself, it should be appreciated that for some physicians, dermal filler treatment makes up the great majority their practice. These physicians, while highly skilled in their field, need to repeat this procedure numerous times throughout the day. Physical and mental fatigue can result, making it difficult to maintain the precision needed for the best aesthetic results.

Devices are needed for facilitating dermal filler injection practice.

### SUMMARY

The present invention provides a device, for example, a syringe or a syringe accessory that greatly facilitates the aspiration procedure described above. Advantageously, the present invention is simple and inexpensive to manufacture and is uncomplicated to use. In some embodiments, the invention provides an accessory that is engageable with an existing dermal filler syringe. Alternatively, the accessory makes up a molded part of a specially designed dermal filler syringe in accordance with the invention. In any event, the device makes it easier for a physician to practice good dermal filler injection techniques on his patients.

More specifically, in one aspect of the invention, a device is provided for facilitating an aspiration procedure (such as described above) during a dermal filler injection treatment. The device makes it possible for a physician to perform the aspiration and injection using the same hand and without requiring a change in his grip.

The device generally comprises an accessory engageable with a dermal filler syringe plunger, the accessory including structure for enabling coupling of the accessory with a proximal end of said syringe plunger. The accessory comprises a thumb stop element structured to engage a thumb moved against the stop element in a proximal direction when the accessory is so coupled to said proximal end of said syringe plunger. Thus, in practice, by using the present device, the physician can move or pull his thumb away from the patient to cause the aspiration of the syringe, and can move his thumb toward the patient to cause the injection of the filler. The structure for enabling coupling may comprise a snap structure, or any suitable means for connecting the device to a syringe plunger, for example, to a thumb grip of a syringe plunger.

In some embodiments, the proximal portion comprises a loop, for example, a closed loop. The stop element may be a distal region of the loop. Alternatively, the proximal portion comprises a hook, and the stop element is a distal region of the hook.

Each and every feature described herein, and each and every combination of two or more of such features, is included within the scope of the present invention provided that the features included in such a combination are not mutually inconsistent.

### BRIEF DESCRIPTION OF THE DRAWINGS

The present invention may be more clearly understood and the advantages and features thereof be more readily appreciated when considering the following detailed description and accompanying drawings of which:

FIG. 1 is a perspective view of a syringe in accordance with an embodiment of the invention, as it is being used during a dermal filler treatment;

FIG. 2 is another perspective view of the syringe being held by an operator's hand;

FIG. 3 is a top perspective view of a component or accessory of the syringe shown in FIGS. 1 and 2;

FIG. 4 is a bottom perspective view of the component or accessory shown in FIG. 3;

FIG. 5 is a side view of the syringe shown in FIGS. 1 and 2;

FIG. 6 is a side view of an alternative embodiment of the invention; and

FIG. 7 is a side view of yet another alternative embodiment of the invention.

### DETAILED DESCRIPTION

The present invention is advantageously designed to allow a physician/injector to aspirate and inject using only a single hand, or aspirate using two hands without having to change his grip on a dermal filler syringe. In one aspect of the invention, an aspiration accessory is provided which may be attached to the plunger rod of the syringe intended for patient injection, prior to use of the syringe on the patient. Once the accessory is attached, the physician can manipulate the syringe for injection of product just as he normally would. When the needle and syringe are at the desirable injection site, the physician will then use their injection thumb to pull back on the plunger to test for vessel proximity. If no blood is detected in the syringe then the physician will then use the same thumb to inject the desired volume of product at the target needle location.

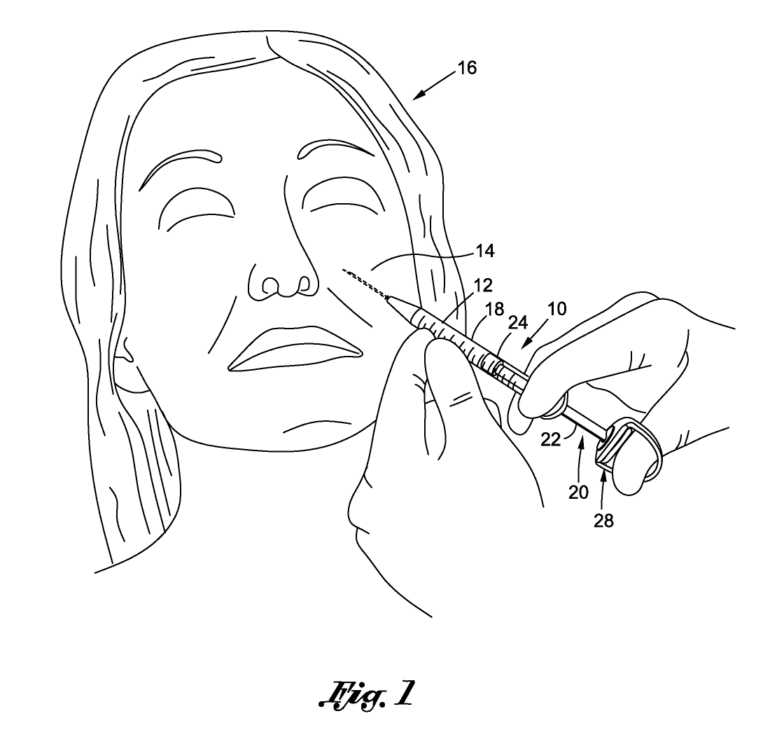

Turning now to FIG. 1, a dermal filler syringe in accordance with an embodiment of the invention is shown generally at 10. The syringe 10 is depicted as being used during a dermal filler treatment in which a dermal filler, for example, a hyaluronic acid-based dermal filler, is introduced into soft tissue, for example, to add volume to sunken cheeks 14 of a patient 16.

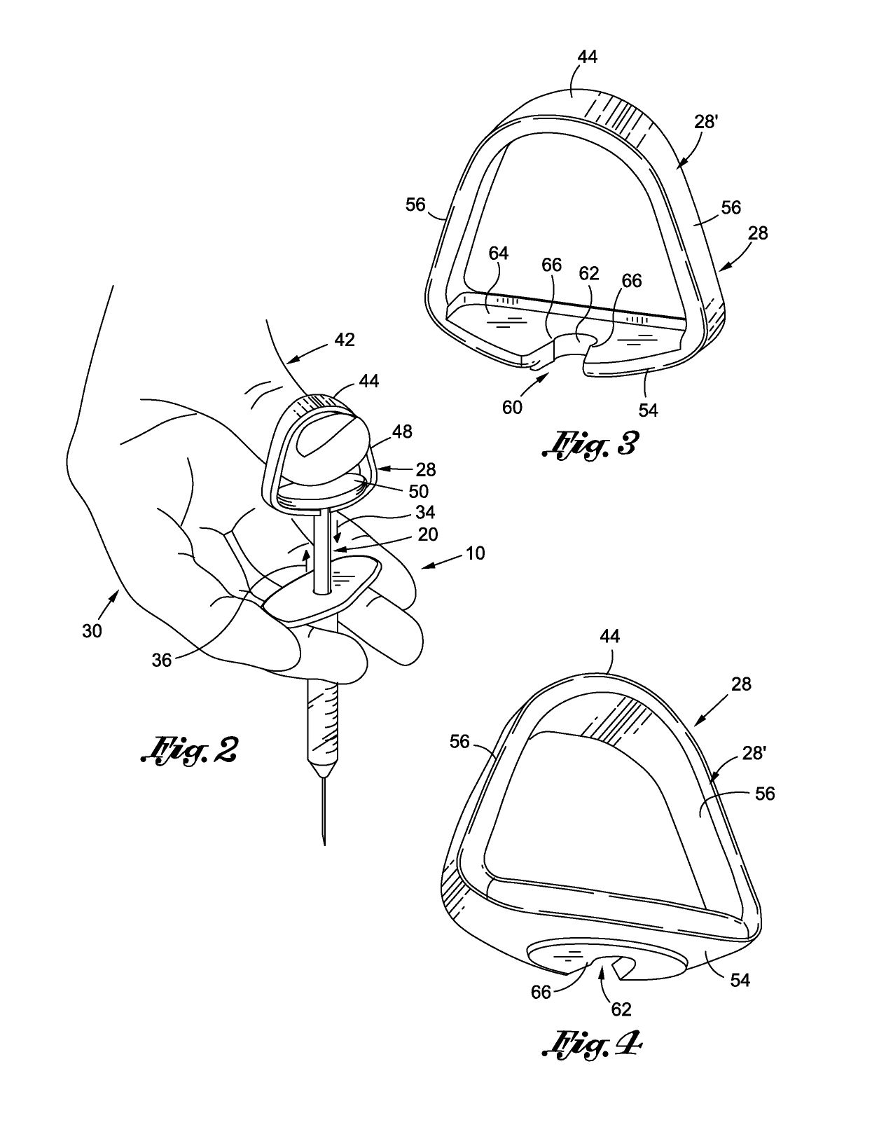

Referring as well to FIG. 2, the syringe 10 generally comprises a barrel 18 for containing an injectable material 12, for example, an injectable gel, for example, an injectable, hyaluronic acid-based dermal filler material. The syringe 10 further includes a plunger 20 slidable in the barrel 18. The plunger 20 includes a rod 22, a distal portion 24 for contacting the injectable material 12 in the barrel 18, and a proximal portion 28.

In FIG. 2, the syringe 10 is shown as being held by a right hand 30 of an operator, e.g. a physician. The proximal portion 28 of the plunger 20 is structured to facilitate manually-induced movement of the plunger 20 in both a distal direction (indicated by arrow 34) and a proximal direction (indicated by arrow 36) using only single digit of the operator's hand 30, most typically the thumb 42, when the syringe 10 is gripped with the hand 30, for example, between the middle finger and ring finger of the hand.

For example, the proximal portion 28 comprises a thumb stop element 44 which is structured to engage the thumb 42 when the thumb 42 is moved against the stop element 44 in the proximal direction 36.

In this specific embodiment, the proximal portion 28 comprises a loop 48, and the stop element 44 makes up a proximal region of the loop 48, for example, the inside surface thereof . The loop 48 may be sized to contain an adult sized thumb, while leaving ample space to allow insertion and removal of the thumb without difficulty. For example, the loop 48 may have an inner diameter or spacing of between about 20 mm and 50 mm. The loop may be available in various sizes to suit different physicians. For example, smaller loops may be provided for female physicians and larger loops may be provided for male physicians.

In the embodiment shown in FIGS. 1-4, the loop 48 is a closed loop. However, turning briefly to FIG. 7, other configurations, such as open-configurations, such as a hook-like configuration, are contemplated and are considered to be within the scope of the invention.

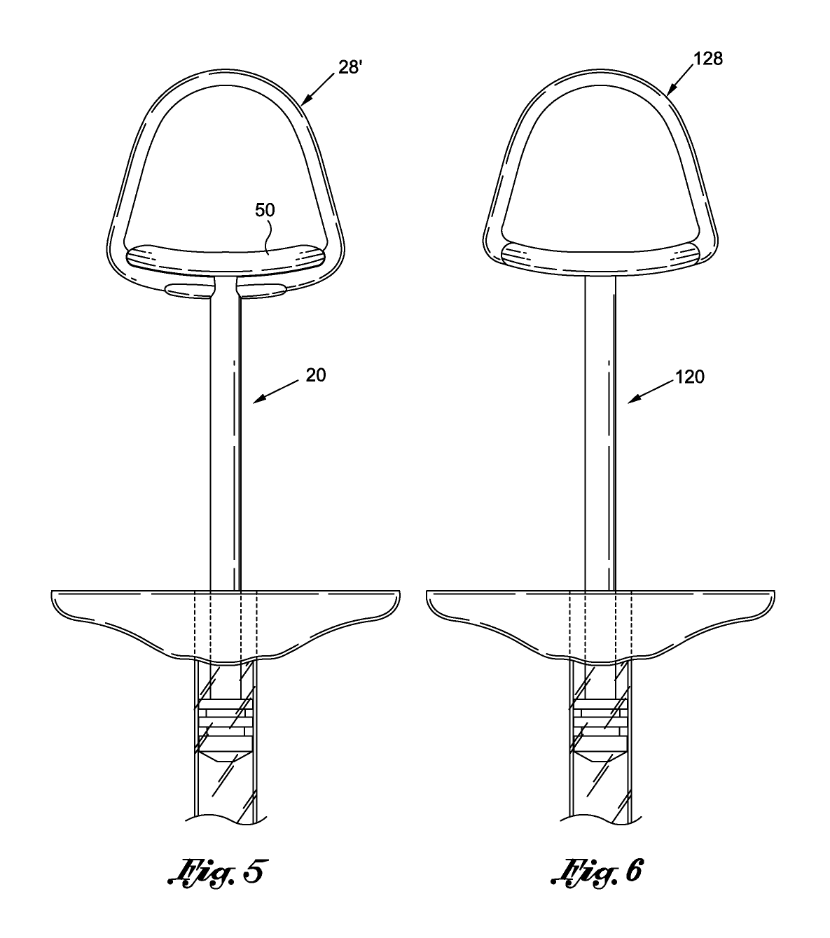

The proximal portion 28 may be in the form of a separate component or accessory 28′ that is engageable or couplable with a dermal filler syringe plunger, for example a conventional dermal filler syringe plunger. For example, the plunger 20 may include a thumb grip 50, for example, a conventionally shaped, syringe thumb grip. Accessory 28′ includes structure effective to enable a simple but effective attachment between a distal region 54 of the proximal portion 28 and the syringe plunger thumb grip 50. Distal region 54 of accessory 28′ may be a flattened region as shown, having a width slightly larger than the thumb grip 50. Opposing sidewalls 56 extend toward proximal region 44. Sidewalls 56 may function in part to provide security to the thumb 42, maintaining the placement of the thumb, for example, during the distal and proximal movements of the thumb during the aspiration and injection steps. Structure 60 may be a snap structure, in that accessory 28′ is snapped into place on thumb grip/rod and provides a solid, secure connection therebetween. FIGS. 3 and 4 illustrate exemplary structure that enables accessory 28′ to be coupled with thumb grip 50, using a simple, snap engagement mechanism. For example, structure 60 may include recessed region 62, shaped to receive rod 22, and recessed seat 64 configured to receive thumb grip 50. Recessed region may have a tapered profile with shoulders 66 to provide a secure grip between accessory 28′ and plunger rod 22.

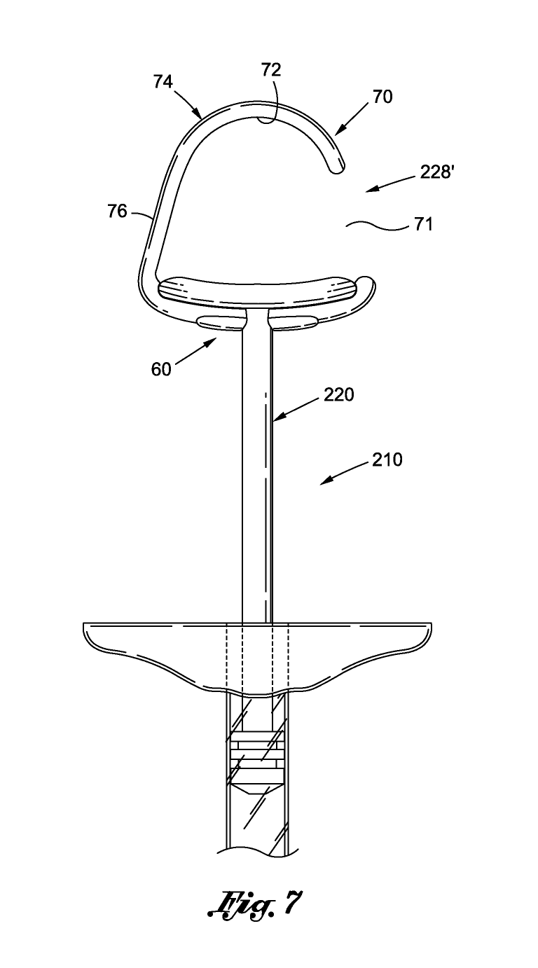

As briefly mentioned hereinabove, in other embodiments of the invention, proximal portion 28 is in the form of a component 128 molded into, or as a part of, the plunger/rod, as shown in FIG. 6.

Turning briefly to FIG. 7, a syringe in accordance with another embodiment of the invention is shown generally at 210. Syringe 210 may be substantially the same as syringe 10, with the major difference being that accessory 228′ in the form of a hook 70 or other “open” configuration, for example, a C-shaped configuration, is provided. This configuration is an alternative embodiment, which functions similarly to accessory 28′. Open portion 71 of hook 70 allows for ease of thumb insertion and removal. In this case, stop element 72 makes up a proximal region 74 of the hook 70. Sidewall 76 connects stop element 72 with engagement structure 60, such as described above and shown in FIG. 3-4. Alternatively, as mentioned above, accessory 228′ may be molded into, or an integral part of, plunger 220.

While this invention has been described with respect to various specific examples and embodiments, it is to be understood that the invention is not limited thereto and that it can be variously practiced within the scope of the invention.

What is claimed is:

1. A dermal filler syringe comprising:
a barrel for containing a dermal filler material; and
a plunger slidable in the barrel;
the plunger including a rod, a distal portion for contacting dermal filler in the barrel, and a proximal portion comprising a thumb stop element structured to engage a thumb moved against the stop element in a proximal direction.

2. The syringe of claim 1 wherein the proximal portion comprises a loop.

3. The syringe of claim 2 wherein the stop element is a distal region of the loop.

4. The syringe of claim 1 wherein the loop is a closed loop.

5. The syringe of claim 1 wherein the proximal portion comprises a hook.

6. The syringe of claim 5 wherein the stop element is a distal region of the hook.

7. A device for facilitating an aspiration procedure during a dermal filler injection treatment, the device comprising:
an accessory engageable with a dermal filler syringe plunger, the accessory including structure for enabling coupling of the accessory with a proximal end of said syringe plunger;
the accessory comprising a thumb stop element structured to engage a thumb moved against the stop element in a proximal direction when the accessory is so coupled to said proximal end of said syringe plunger.

8. The device of claim 7 wherein the structure for enabling coupling comprises a snap structure.

9. The syringe of claim 7 wherein the proximal portion comprises a loop.

10. The syringe of claim 9 wherein the stop element is a distal region of the loop.

11. The syringe of claim 7 wherein the loop is a closed loop.

12. The syringe of claim 7 wherein the proximal portion comprises a hook.

13. The syringe of claim 12 wherein the stop element is a distal region of the hook.
